(19) United States
(12) Patent Application Publication
(10) Pub. No.: US 2010/0203621 A1
(43) Pub. Date: Aug. 12, 2010

(54) BIOSENSOR

(75) Inventors: Makoto Takahashi, Osaka (JP); Masaya Nakatani, Hyogo (JP); Hiroshi Ushio, Osaka (JP)

Correspondence Address: RATNERPRESTIA, P.O. BOX 980, VALLEY FORGE, PA, 19482, US

(73) Assignee: Panasonic Corporation, Kadoma-shi ,Osaka (JP)

(21) Appl. No.: 12/670,206

(22) PCT Filed: Jul. 14, 2008
(86) PCT No.: PCT/JP2008/001880

§ 371 Date: Jan. 22, 2010

(30) Foreign Application Priority Data
Jul. 25, 2007 (JP) 2007-192805
Aug. 21, 2007 (JP) 2007-214327

(51) Int. Cl. C12M 1/34

(52) U.S. Cl. 435/287.1

(57) ABSTRACT

A biosensor has the following elements: a diaphragm having a through-hole; a frame supporting the diaphragm and having a cavity; and pillars formed on the side of inner wall surface of the frame and on the surface of the diaphragm. This structure can suppress the occurrence of bubbles in the vicinity of the through-hole, and easily remove the remaining bubbles. Thus the measuring reliability of the biosensor can be improved.

This Application is a U.S. National Phase Application of PCT International Application PCT/JP2008/001880.

### TECHNICAL FIELD

The present invention relates to a biosensor, such as a cell electrophysiological sensor.

### BACKGROUND ART

In recent years, a micro biosensor to which microelectromechanical systems (MEMS) technology is applied has been drawing attention. An example of a biosensor is a cell electrophysiological sensor. This cell electrophysiological sensor is used in an automation system of a patch clamp method for clarifying the function of ion channels present in a cell membrane using the electrical activity of the cell as an index, or screening (testing) a medicine.

With reference to FIG. 14, a conventional biosensor (cell electrophysiological sensor) disclosed in Patent Literature 1 is described. In FIG. 14, conventional biosensor 31 has diaphragm 32, and recess 33 formed in the top surface of diaphragm 32. The biosensor also has through-hole 34 connecting the bottom portion of recess 33 and the bottom surface of diaphragm 32, reference electrode 35 disposed above diaphragm 32, and measuring electrode 36 disposed inside of through-hole 34. Measuring electrode 36 is coupled to a signal detector via wiring 37. Diaphragm 32 is disposed inside of well 38.

In biosensor 31, first, a cell and electrolytic solution 40 are injected into well 38. The cell is trapped (captured) by recess 33, and held onto the opening of through-hole 34. The cell held by recess 33 is referred to as specimen cell 39 hereinafter. In this conventional biosensor 31, recess 33 and through-hole 34 form a holder of specimen cell 39.

During measurement, specimen cell 39 is sucked with a suction pump, for example, from the downward direction of through-hole 34, and held onto the opening of through-hole 34 in intimate contact therewith. That is, through-hole 34 has a role similar to that of the tip hole of a glass pipette. The functionality and pharmacodynamic reaction of the ion channels in specimen cell 39 are analyzed by measuring the voltage or current between reference electrode 35 and measuring electrode 36 before and after the reaction and obtaining the difference in potential between the inside and outside of the cell.

However, such a conventional biosensor 31 has errors in the measurements of the potential difference between reference electrode 35 and measuring electrode 36, so that the measuring reliability of biosensor 31 is degraded.

This is caused by the presence of bubbles in the vicinity of the holder of specimen cell 39. The resistance of the bubbles is so large that the presence of the bubbles causes variations in measurements. As a result, the measuring reliability of biosensor 31 is degraded.

[Patent Literature 1] International Publication No. 02/055653 Pamphlet

### SUMMARY OF THE INVENTION

The present invention is directed to provide a biosensor that has reduced measuring errors and improved measuring reliability.

The biosensor of the present invention has the following elements:
a diaphragm having a specimen holder;
a frame supporting the diaphragm and having a cavity; and
pillars formed on the side of the inner wall surface of the frame and on the surface of the diaphragm.

This structure can suppress the occurrence of bubbles in the vicinity of the specimen holder, and efficiently remove the remaining bubbles. Thus the measuring reliability of the biosensor can be improved.

### BRIEF DESCRIPTION OF DRAWINGS

FIG. 1 is a perspective view of a cell electrophysiological sensor in accordance with a first exemplary embodiment of the present invention.

FIG. 2 is a top view of the cell electrophysiological sensor.

FIG. 3 is a sectional view taken on line 3-3 of FIG. 2.

FIG. 4 is a sectional view of a cell potential measuring device that includes the cell electrophysiological sensor in accordance with the first exemplary embodiment.

FIG. 5 is a sectional view of a cell electrophysiological sensor in accordance with a second exemplary embodiment of the present invention.

FIG. 6 is a sectional view of a cell electrophysiological sensor in accordance with a third exemplary embodiment of the present invention.

FIG. 7 is a perspective view of a cell electrophysiological sensor in accordance with a fourth exemplary embodiment of the present invention.

FIG. 8 is a sectional view of the cell electrophysiological sensor.

FIG. 9 is a sectional view of a cell potential measuring device that includes the cell electrophysiological sensor in accordance with the fourth exemplary embodiment.

FIG. 10 is a sectional view of a cell electrophysiological sensor in accordance with a fifth exemplary embodiment of the present invention.

FIG. 11 is a sectional view of a cell electrophysiological sensor in accordance with a sixth exemplary embodiment of the present invention.

FIG. 12 is a top view of a cell electrophysiological sensor in accordance with a seventh exemplary embodiment of the present invention.

FIG. 13 is a sectional view of a cell electrophysiological sensor in accordance with an eighth exemplary embodiment of the present invention.

FIG. 14 is a sectional view of a conventional extracellular potential measuring sensor.

### REFERENCE MARKS IN THE DRAWINGS

1 Through-hole
2 Diaphragm
3 Frame
4 Cavity
5 End surface
6 Pillar
9 Cell
9a Specimen cell
10 Extracellular fluid
11 Intercellular fluid
12 Reference electrode
13 Measuring electrode
14 Baffle plate
14a Opening
15 Well layer
15a Well
16 Fluid channel
17 Case
17a Plate
18 Projections and depressions
19 Groove
20 Cell electrophysiological sensor
21 Ridge line

### DETAILED DESCRIPTION OF PREFERRED EMBODIMENTS

Hereinafter, a biosensor of the present invention is detailed, using a cell electrophysiological sensor as an example, with reference to the accompanying drawings.

### First Exemplary Embodiment

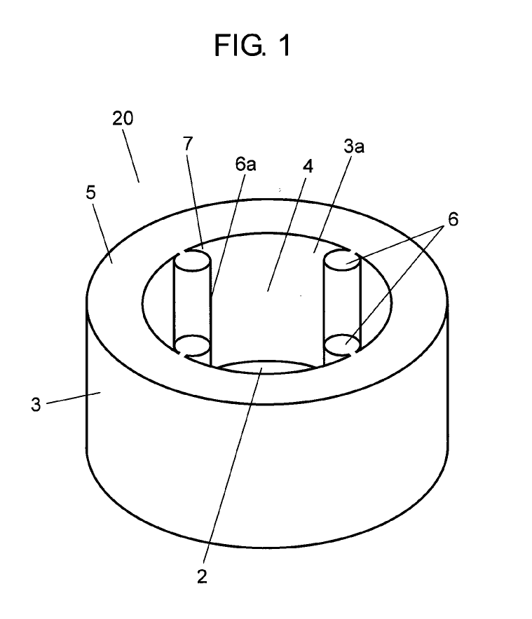

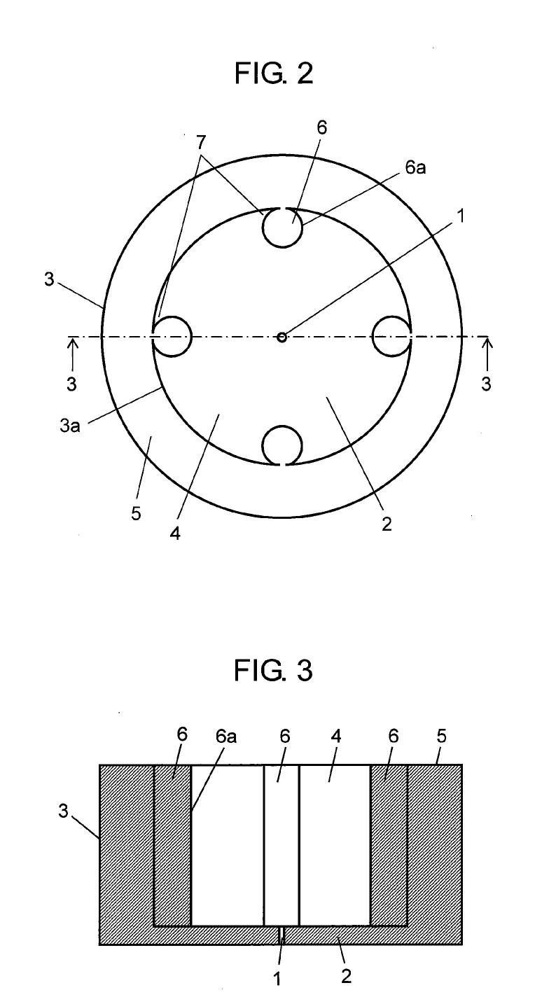

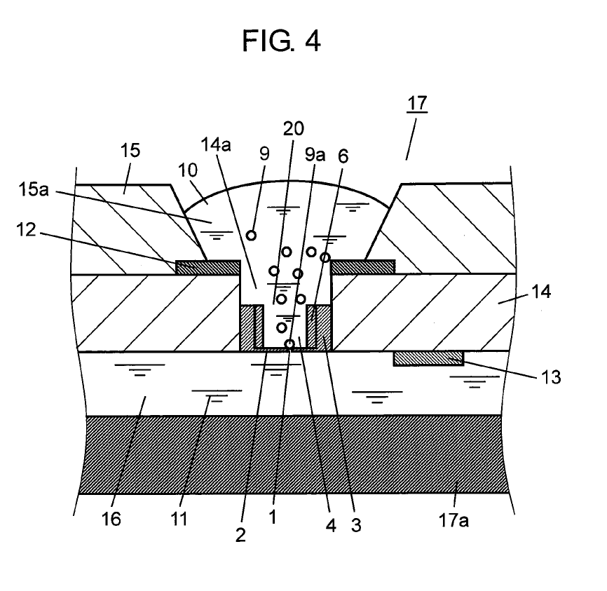

FIG. 1 is a perspective view of a cell electrophysiological sensor in accordance with the first exemplary embodiment of the present invention. FIG. 2 is a top view of FIG. 1. FIG. 3 is a sectional view taken on line 3-3 of FIG. 2. FIG. 4 is a sectional view of a cell potential measuring device for explaining a method for measuring an electrophysiological phenomenon of cells.

First, a description is provided for the structure of the cell electrophysiological sensor of this exemplary embodiment. With reference to FIG. 1 through FIG. 3, cell electrophysiological sensor 20 of this exemplary embodiment has thin plate-shaped diaphragm 2 having at least one through-hole 1, and frame 3 for fixedly supporting diaphragm 2 and facilitating fixation of the sensor to a measuring device, for example. Through-hole 1 captures and holds a cell, i.e. a specimen, and serves as a specimen holder.

At least one through-hole 1 is sufficient. In order to measure a plurality of cells 9 (see FIG. 4) at the same time, a plurality of through-holes 1 can be formed. With this structure, the plurality of cells 9 can be measured at the same time to improve the S/N ratio.

Cavity 4 capable of pooling a liquid that contains cells, for example, is formed inside of frame 3. Further, pillars 6 each having a circular sectional shape is formed unitarily with frame 3 in contact with inner wall surface 3a of frame 3, on the surface of diaphragm 2. Inner wall surface 3a of frame 3 and outer wall surface 6a of each pillar form an acute angle and recess 7 therebetween.

The sensor has recesses 7 formed by inner wall surface 3a of frame 3 and outer wall surfaces 6a of the pillars. As will be described later, this structure suppresses the occurrence of bubbles and efficiently removes the remaining bubbles when a liquid in the form of droplets is injected into cavity 4 with a dispenser, for example, in a manner described in the conventional art. At this time, when the sectional shape of each pillar 6 is a circular or oval shape, recess 7 where outer wall surface 6a of the pillar forms an acute angle with inner wall surface 3a of the frame can be formed easily.

Further, setting the height of pillar 6 equal to the height of end surface 5 of frame 3 allows the cell electrophysiological sensor to be processed and handled easily.

It is preferable in terms of processability, mechanical strength, or the like, that the thickness of diaphragm 2 ranges from 10 to 300 μm. It is preferable in terms of productivity that the inner diameter of cavity 4 ranges from 100 μm to 1.0 mm. When the inner diameter of cavity 4 is smaller than 100 μm, cells cannot be dispensed easily, and many bubbles remain. When the inner diameter of cavity 4 exceeds 1 mm, the productivity of the sensor is reduced. Further, when a liquid containing a small amount of cells 9 is measured, the bottom surface is too large. This lowers the probability that cells 9 are captured in through-hole 1, and is not preferable.

In this exemplary embodiment, wafers of silicon, for example, having high obtainability and processability, are prepared. Then, by a general etching process using a photolithography technique, for example, a plurality of cell electrophysiological sensors 20 are produced collectively. With this method, cell electrophysiological sensors having high dimensional accuracy and microshapes can be produced efficiently.

Diaphragm 2, frame 3, and pillars 6 of this exemplary embodiment are unitarily formed from a single-crystal silicon substrate by dry etching. With this method, pillars 6 are unitarily formed with inner wall surface 3a of the frame, so that micro pillars 6 can be formed stably.

In this exemplary embodiment, the specimen holder is through-hole 1, and thus this through-hole 1 can also be formed by the dry etching process. Therefore, through-hole 1, diaphragm 2, frame 3, and pillars 6 can be formed in one process with high productivity.

In this exemplary embodiment, a single-crystal silicon substrate is used. A so-called SOI (silicon on insulator) substrate formed by vertically sandwiching an SiO₂ layer between silicon layers can also be used. In this case, one silicon layer is etched until the SiO₂ layer is exposed to form frame 3, and the laminate of the SiO₂ layer and the other silicon layer can be used as diaphragm 2. Then, the opening of through-hole 1 is formed in the surface of the SiO₂ layer. Thus the surface of the SiO₂ layer having high hydrophilic properties forms a cell holding surface, and is capable of holding a cell in intimate contact therewith.

A description is provided for a structure of a cell potential measuring device for measuring electrophysiological activity of cells that includes cell electrophysiological sensor 20 of this exemplary embodiment structured as above.

FIG. 4 is a sectional view of an essential part showing a cell potential measuring device that includes cell electrophysiological sensor 20 in accordance with this exemplary embodiment.

With reference to FIG. 4, case 17 made of an insulator, such as plastics, has baffle plate 14 for partitioning the inside of case 17 into a top part to which extracellular fluid 10 is supplied, and a bottom part to which intracellular fluid 11 is supplied. Baffle plate 14 has a plurality of openings 14a formed in a matrix shape. Inside of each opening 14a, cell electrophysiological sensor 20 as described above is set. In the layer above baffle plate 14, well 15a for pooling a liquid and well layer 15 are formed so as to correspond to each opening 14a.

In terms of productivity, processability, and dimensional accuracy, it is preferable that baffle plate 14 and well layer 15 are formed of resin.

Cell electrophysiological sensor 20 is joined to the inside of opening 14a without gaps so that diaphragm 2 is on the bottom side and no fluid leaks into opening 14a. Thus the space inside of case 17 is partitioned into two vertical areas by diaphragm 2 and baffle plate 14a. The two vertical areas partitioned by baffle plate 14 separately pool a liquid, e.g. extracellular fluid 10 and culture solution, and a liquid, e.g. intercellular fluid 11 and medical solution. Extracellular fluid 10 includes a liquid that contains cells 9 or the like. Among a plurality of cells 9 suspended in extracellular fluid 10, cell 9 is sucked with a suction mechanism, such as a suction pump, from the downward direction of baffle plate 14, and fixed to through-hole 1 so as to block the through-hole. Such a cell is specimen cell 9a. Thus the liquid above these specimen cells 9a and the liquid below the cells move only via through-holes 1. In this exemplary embodiment, the appropriate suction force of the suction mechanism ranges from 2 kPa to 10 kPa. The suction force is set to different values in accordance with the length of through-hole 1.

Case 17 of this exemplary embodiment has a three-layer structure: plate 17a having fluid channel 16 formed as described above; baffle plate 14 joined onto plate 17a; and well layer 15 joined onto baffle plate 14. Case 17 may be formed by bonding the above three members, or integrally molded by injection molding, for example. Alternatively, for example, only well layer 15 and baffle plate 14 are integrally molded, and plate 17a having fluid channel 16 is bonded to the integrally molded part.

A liquid, e.g. intercellular fluid 11 and medical solution, is charged into or removed from fluid channel 16 with a fluid delivery mechanism, such as a micropump.

Further, on the top side of baffle plate 14, reference electrode 12 made of silver, silver chloride, or the like is disposed in extracellular fluid 10. On the bottom side of baffle plate 14, measuring electrode 13 similarly made of silver, silver chloride, or the like is disposed in intercellular fluid 11.

These reference electrode 12 and measuring electrode 13 may be interchanged with each other. Reference electrode 12 and measuring electrode 13 can be made of a material selected from chromium, titanium, copper, gold, platinum, silver, and silver chloride. As reference electrode 12 and measuring electrode 13, a needle-shaped microelectrode probe may be used.

Next, a description is provided for a method for measuring cell potential, using the cell potential measuring device structured as above.

First, a predetermined amount of extracellular fluid 10, i.e. a liquid containing cells 9 dispersed therein, is injected into cavity 4, using an automatic dispenser, for example. Generally, in this case, the liquid has a spherical appearance and shape formed by surface tension. The liquid is dispensed into cavity 4 in that shape.

At this time, in order to measure electrochemical changes in specimen cell 9a stably with high accuracy, it is important to charge the liquid without bubbles remaining on diaphragm 2, especially in the vicinity of through-hole 1.

When end surface 5 (see FIG. 1) of frame 3 of cell electrophysiological sensor 20 is formed of one cylindrical plane without pillars 6, i.e. in a structure without recesses 7, a droplet at the tip of a micropipette dispensed from the dispenser can completely block end surface 5 of frame 3 in some cases. This makes the inside of cavity 4 hermetically sealed. Thus the gas, such as air, present inside of cavity 4 has no escape route and remains as bubbles during measurement. Further, the lack of the escape route hinders smooth injection of droplets into cavity 4.

When bubbles remain on the surface of diaphragm 2, especially in the vicinity of through-hole 1, i.e. the specimen holder, in this manner, it is difficult to accurately measure microchanges in voltage or current, which are electrochemical changes in specimen cell 9a fixed onto through-hole 1 with the suction mechanism.

In contrast, in this exemplary embodiment, as shown in FIG. 1, pillars 6 are formed on the surface of diaphragm 2 in contact with inner wall surface 3a of frame 3, thus forming recesses 7 where inner wall surface 3a of frame 3 forms an acute angle with outer wall surface 6a of each pillar 6. With this structure, when a droplet in a spherical shape makes contact with end surface 5 of frame 3, the tip of the droplet makes contact with the tips of pillars 6 by means of the surface tension of the droplet. Thus, while the droplet is wetting outer wall surfaces 6a of the pillars and recesses 7, the liquid made of extracellular fluid 10 can be promptly charged into cavity 4 together with cells 9 without bubbles occurring therein. The remaining bubbles can be pushed out from recesses 7 and removed in the upward direction.

Preferably, as the sectional shape, each pillar 6 acting as above has a diameter equal to or larger than 100 μm. A pillar having a diameter smaller than 100 μm is easily broken and has a small surface area, so that the droplet cannot be charged efficiently. A diameter larger than 100 μm is restricted by the size of cavity 4. In order to make such an action more effective, it is preferable to form a plurality of pillars 6. This structure allows more efficient permeation of the liquid by means of surface tension and efficient removal of remaining bubbles.

Further, the above action can be enhanced by forming a plurality of recesses 7 at equal spacings in a radial configuration around through-hole 1 at the center. That is, this structure can ensure the contact of a part of the droplet with a part of pillars 6 and guide the flow of the droplet to recesses 7 even when the tip of the dispenser, such as a micropipette, tilts or the droplet has a less symmetrical shape. Thus, when a plurality of recesses 7 are formed, the liquid, such as extracellular fluid 10, can be reliably charged from outer wall surfaces 6a of the pillars and recesses 7 formed on the surface of diaphragm 2 while the occurrence of bubbles is suppressed.

In the above structure, pillars 6 also act as reinforcing beams. Thus the thickness of frame 3 can be reduced and the cell electrophysiological sensor 20 can be downsized.

Further, imparting hydrophilic properties to outer wall surfaces 6a of the pillars enhance the wettability of outer wall surfaces 6a of the pillars to the liquid, and allow the liquid to be charged into cavity 4 promptly. At the same time, the liquid can be charged with fewer bubbles remaining on the surface of diaphragm 2.

In this case, in order to impart hydrophilic properties to outer wall surfaces 6a of the pillars, it is preferable to remove carbon molecules adhering to the surfaces so that the surfaces are made clean. Immediately thereafter, the frame is stored in a container filled with pure water. Thereby, the cleanliness of the surfaces can be maintained. Preferably, the hydrophilic properties at this time are such that the contact angle is equal to or smaller than 10°. This contact angle can be measured by applying a droplet of de-ionized pure water to the surface of an object to be measured.

Further, as another means for enhancing hydrophilic properties, coating the surfaces of outer wall surfaces 6a of the pillars with an insulating film of silicon dioxide, for example, is effective.

In this manner, in the cell electrophysiological sensor of this exemplary embodiment, pillars 6 are formed in contact with inner wall surface 3a of the frame, thus forming recesses 7 so that inner wall surface 3a of the frame forms an acute angle with outer wall surface 6a of each pillar. With this structure, while wetting outer wall surfaces 6a of the pillars and recesses 7, the liquid can be charged into cavity 4 promptly. At the same time, the gas, such as air, remaining inside of cavity 4 can be released to the outside easily. Thus the liquid can be charged without bubbles on the surface of diaphragm 2.

In this manner, in this exemplary embodiment, a liquid can be charged into cell electrophysiological sensor 20 without remaining bubbles as shown in FIG. 4.

Thereafter, using a suction mechanism, such as a suction pump, a predetermined pressure difference is caused between the top and bottom sides of baffle plate 14 so that the bottom side of baffle plate 14 has a lower pressure. At this time, one cell 9 is attracted to the opening of through-hole 1 and trapped onto through-hole 1. Cell 9 trapped onto through-hole 1 is specimen cell 9a. When this pressure difference is maintained, sufficient adherence is ensured, and thus an electrical resistance is provided between extracellular fluid 10 and intercellular fluid 11 in fluid channel 16.

Specifically, when one side of fluid channel 16 is sealed and the fluid channel is depressurized from the other side, cell 9 is attracted to through-hole 1. At last, specimen cell 9a blocks through-hole 1, and is trapped therein. With this operation, the electrical resistance between cavity 4 filled with extracellular fluid 10 and fluid channel 16 filled with intercellular fluid 11, for example, is sufficiently increased.

The liquid to be charged is not specifically limited. Besides extracellular fluid 10, a culture solution for culturing cells 9, or other chemical solutions may be used.

Next, the suction force of the suction mechanism is controlled so that depressurization is continued until a part of the membrane of specimen cell 9a is broken. Further, from the bottom side of through-hole 1, intercellular fluid 11 is introduced into specimen cell 9a. This operation can bring a part of specimen cell 9a into contact with intercellular fluid 11, and specimen cell 9a into a state where its electrochemical changes can be measured.

Alternatively, while specimen cell 9a is securely trapped in through-hole 1, a medical solution acting to dissolve the outer wall of specimen cell 9a, such as nystatin, is introduced into fluid channel 16. With this operation, a microhole is formed in specimen cell 9a, so that a part of specimen cell 9a can be brought into contact with intercellular fluid 11. Therefore, as similar to the above, specimen cell 9a is brought into a state where its electrochemical changes can be measured.

Here, for mammal muscle cells, for example, extracellular fluid 10 is an electrolytic solution that typically contains approximately 4 mM of K⁺ ions, approximately 145 mM of Na⁺ ions, and approximately 123 mM of Cl⁻ ions. For mammal muscle cells, for example, intercellular fluid 11 is an electrolytic solution that typically contains approximately 155 mM of K⁺ ions, approximately 12 mM of Na⁺ ions, and approximately 4.2 mM of Cl⁻ ions.

Incidentally, in a state where well 15a and opening 14a are filled with extracellular fluid 10 (in a state where specimen cell 9a is not trapped in through-hole 1), a resistance in the order of 100 kΩ to 10 MΩ can be observed between reference electrode 12 disposed in well 15a and measuring electrode 13 disposed in fluid channel 16. This is because the electrolytic solutions permeate into through-hole 1 formed in cell electrophysiological sensor 20 and an electrical circuit is formed between reference electrode 12 and measuring electrode 13. That is, this resistance shows that through-hole 1 is properly formed and thus electrical continuity is maintained between the top and the bottom sides of through-hole 1.

Next, changes in current or voltage are measured between reference electrode 12 and measuring electrode 13, so that electrochemical changes in specimen cell 9a are measured and detected. For stable measurement and detection, a state where specimen cell 9a is securely trapped in through-hole 1 needs to be kept stable. For this purpose, preferably, through-hole 1 has a shape slightly smaller than that of specimen cell 9a so that specimen cell 9a can be fixed onto through-hole 1 so as to block this through-hole, with a suction mechanism, for example. For this purpose, in this exemplary embodiment, the diameter of through-hole 1 is set to 3 μm.

As described above, when the size of cell 9 ranges from approximately 5 to 50 μm, in order to keep high adherence between cell 9 and through-hole 1, it is preferable to set the diameter of through-hole 1 to 3 μm or smaller. However, the optimum size of through-hole 1 can be determined in accordance with the shape and nature of cells 9 to be measured.

Next, a stimulation of a chemical compound, such as a medicine, is given to specimen cell 9a. When such stimulation is given, specimen cell 9a shows an electrophysiological response. As a result, between reference electrode 12 and measuring electrode 13, an electrochemical change can be observed as an electrical response in voltage or current, for example.

The above chemical stimulations are caused by chemicals, poisons, or the like. Besides these stimulations, physical stimulations are caused by mechanical modification, light, heat, electricity, electromagnetic waves, or the like.

When specimen cell 9a actively reacts to these stimulations, specimen cell 9a releases or absorbs various ions through ion channels present in its cell membrane. Then, ion current through specimen cell 9a is generated, and changes the gradient of potential inside and outside of this specimen cell 9a. That is, this change can be detected by measuring the voltage or current between reference electrode 12 and measuring electrode 13 before and after the reaction.

In this exemplary embodiment, a description has been provided for an example of cell electrophysiological sensor 20 in the cell potential measuring device where diaphragm 2 is disposed on the bottom side for measurement. However, diaphragm 2 can be disposed on the top side for measurement. In this case, the occurrence and remaining of bubbles in intercellular fluid 11 can be suppressed, and specimen cell 9a makes intimate contact with the opening of through-hole 1 on the side opposite to the side in the above description. Such a structure can be used for a case where the intimate contact of specimen cell 9a with a hole formed through a flat surface is convenient. Preferably, which structure to use is determined in accordance with the nature of specimen cell 9a, for each case.

When an electrical response is measured between reference electrode 12 and measuring electrode 13, any bubble present in the vicinity of through-hole 1 increases the resistance, thus causing variations in the measurements of the current or voltage detected in measuring electrode 13. However, cell electrophysiological sensor 20 of this exemplary embodiment structured as shown in FIG. 1 can suppress the occurrence of bubbles and promptly remove the remaining bubbles as described above. Thus microchanges in current or voltage can be measured with high accuracy and the measuring reliability can be improved.

Incidentally, when a liquid is dispensed into cell electrophysiological sensor 20, small bubbles can remain in a part of the junction between diaphragm 2 and recesses 7 in some cases. However, it is known that the small bubbles are distant from through-hole 1, and the presence of such small bubbles has a small influence on measurement.

Preferably, all the surfaces to be in contact with the liquid, e.g. inner wall surface 3a of the frame, the surface of diaphragm 2, and the inner wall surface of through-hole 1, are made hydrophilic. This structure can enhance wettability to the liquid for various measurements, thus suppressing the occurrence and remaining of bubbles more effectively.

Further, when a plurality of recesses 7 are formed so as to have different dimensions and shapes, a sensor structure that facilitates measurement of liquids having different properties, including viscosity and constituents, can be implemented.

Extracellular fluid 10 and intercellular fluid 11 may have different compositions as described in this embodiment, or the same composition.

In this exemplary embodiment, diaphragm 2 has a disk shape. However, diaphragms in other shapes, such as a quadrangular shape, can provide the similar advantage.

As described above, in this exemplary embodiment, a droplet containing cells 9 and dispensed from a dispenser makes contact with a part of the nearest pillar 6. Thus the liquid moves toward diaphragm 2 while flowing toward recesses 7 formed by outer wall surfaces 6a of pillars 6 and inner wall surface 3a of frame 3 by means of the surface tension. Thereafter, the liquid is guided to the center of diaphragm 2 where through-hole 1 is present. Thus the occurrence of bubbles can be suppressed and remaining bubbles can be removed efficiently. Therefore, microchanges in current or voltage can be measured with high accuracy without any influence of bubbles, and the measuring reliability can be improved.

### Second Exemplary Embodiment

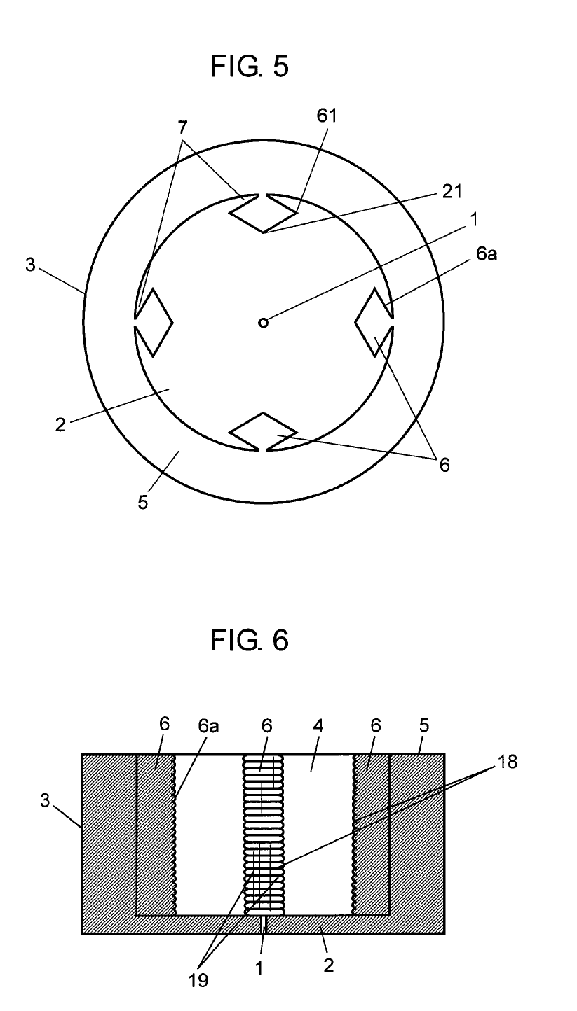

A description is provided for a cell electrophysiological sensor in accordance with the second exemplary embodiment of the present invention with reference to FIG. 5.

FIG. 5 is a top view of the cell electrophysiological sensor in accordance with the second exemplary embodiment of the present invention. In FIG. 5, the structure of the cell electrophysiological sensor of this exemplary embodiment largely differs from that of the cell electrophysiological sensor of the first exemplary embodiment in that the sectional shape of each pillar 6 is a polygonal shape. Thus recesses 7 can be designed into shape patterns in a considerably expanded range. With this structure, in accordance with the shape of the tip of a micropipette, for example, the surface of a droplet can be reliably brought into contact with ridge line 21 or tip corners 61 of each pillar 6. Thus the liquid can be charged promptly and efficiently.

Further, each pillar 6 having a polygonal sectional shape is formed so that ridge line 21 thereof faces through-hole 1. With this structure, a liquid flow is formed along ridge line 21 from the inside to the outside, or the outside to the inside, and the introduced liquid can be stirred and displaced smoothly. Therefore, the liquid flowing from well layer 15 or baffle plate 14 into cavity 4 as shown in FIG. 4 can be stirred and displaced efficiently. As a result, bubbles can escape more effectively.

In this manner, in this exemplary embodiment, the liquid makes contact with tip corners 61 of pillars 6 in a polygonal shape, and thus the liquid can be charged promptly with more reliability.

### Third Exemplary Embodiment

A description is provided for a cell electrophysiological sensor in accordance with the third exemplary embodiment of the present invention with reference to FIG. 6.

FIG. 6 is a sectional view of the cell electrophysiological sensor in accordance with the third exemplary embodiment of the present invention. This exemplary embodiment mainly differs from the first exemplary embodiment in the following points: as shown in FIG. 6, a plurality of arc-shaped projections and depressions 18 are formed on outer wall surface 6a (surface) of each pillar 6 along its side surface, and a plurality of grooves 19 are formed substantially parallel to the long axis direction of pillar 6.

One of the methods for forming projections and depressions 18 is to alternately introduce an etching promoting gas, e.g. SF₆, and an etching inhibiting gas, e.g. C₄F₈, in the presence of plasma, in a dry etching process.

In this manner, projections and depressions 18 substantially perpendicular to the long axis direction of pillar 6 are formed on outer wall surface 6a of pillar 6. In this structure, droplets make contact with a plurality of surfaces in outer wall surface 6a of pillar 6. Then, an attraction force is exerted between the droplets, and the droplets attempt to form a large mass. Thus the liquid can be guided to diaphragm 2 efficiently. Further, the liquid can be spread to the entire outer circumference of outer wall surface 6a of pillar 6 along projections and depressions 18.

When an etching promoting gas is introduced to the outer wall of pillar 6 from an obliquely upward direction, small notches are formed in outer wall 6a. Thereafter, the dry etching process similar to that for forming projections and depressions 18 is continued. Then, grooves 19 starting from the notches can be formed in the long axis direction of pillar 6. Many grooves 19 can be formed by forming many notches.

In this manner, in this exemplary embodiment, a plurality of grooves 19 are formed in the long axis direction of pillar 6. With this structure, a droplet injected from the upward direction can be guided to the bottom side of diaphragm 2 along these grooves 19. That is, the droplets are promptly guided so as not to block the spaces between adjacent pillars 6. This structure allows the cell electrophysiological sensor to ensure an escape route of bubbles, suppress the occurrence of bubbles, and have high measuring reliability. In order to guide the droplets to diaphragm 2, it is effective to form more grooves 19 on the bottom side, i.e. in the vicinity of diaphragm 2.

Further, in this exemplary embodiment, grooves 19 connect a plurality of projections and depressions 18. With this structure, while wetting the outer circumference of each pillar 6, the liquid efficiently flows from the tip side of pillar 6 to the side of diaphragm 2.

In this exemplary embodiment, projections and depressions 18 and grooves 19 increase the surface area, thus increasing the contact surface between a droplet to be injected and pillar 6. Therefore, when outer wall surface 6a of pillar 6 is made hydrophilic by thermal oxidation, for example, the force with which outer wall surface 6a of pillar 6 pulls the droplet is increased, and the liquid can be guided along outer wall surface 6a of pillar 6 to diaphragm 2 where through-hole 1 is present. This structure allows the cell electrophysiological sensor to suppress the occurrence of bubbles and have high measuring reliability.

In this exemplary embodiment, through-hole 1, diaphragm 2, frame 3, pillars 6, projections and depressions 18, and grooves 19 on outer wall surfaces 6a of the pillars can be formed in one process with high productivity.

Projections and depressions 18 do not need to be perpendicular to the long axis direction of pillar 6 as shown in FIG. 6. It is sufficient that the projections and depressions intersect with the long axis direction of pillar 6 to an extent a droplet can be spread to outer wall surface 6a of pillar 6. Grooves 19 do not need to correspond exactly to the long axis direction of pillar 6 as shown in FIG. 6. It is sufficient that the grooves are substantially parallel to the long axis direction of pillar 6 to an extent that a droplet can be guided to the downward direction.

### Fourth Exemplary Embodiment

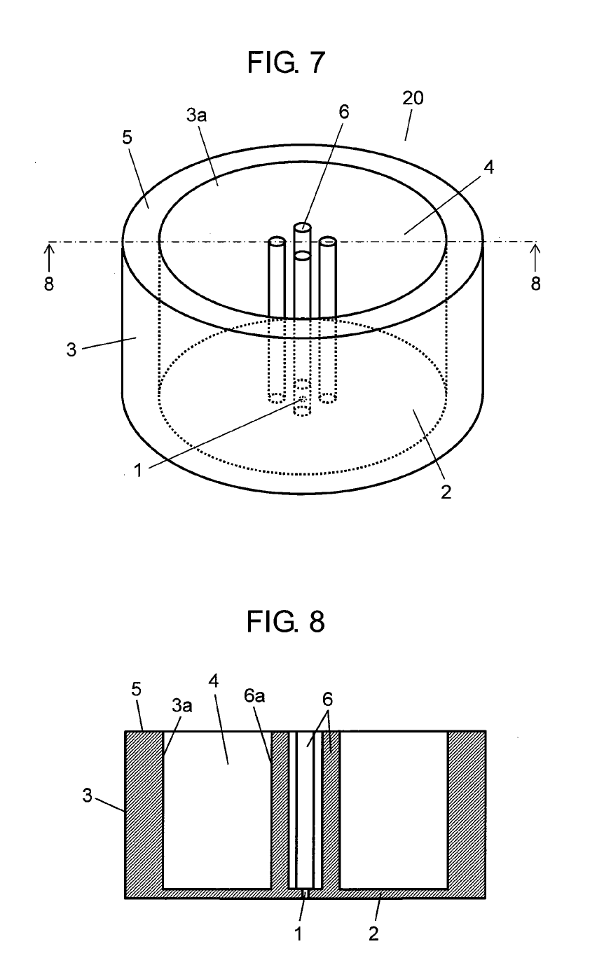

A description is provided for a cell electrophysiological sensor in accordance with the fourth exemplary embodiment of the present invention with reference to FIG. 7 through FIG. 9.

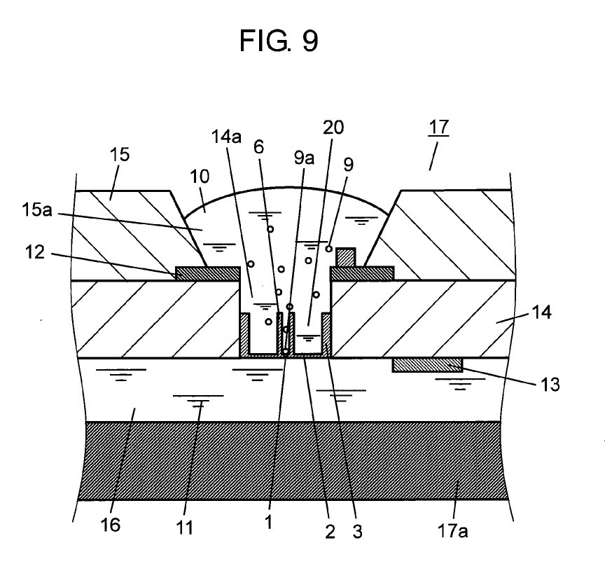

FIG. 7 is a perspective view of the cell electrophysiological sensor in accordance with the fourth exemplary embodiment of the present invention. FIG. 8 is a sectional view taken on line 8-8 of FIG. 7. FIG. 9 is a sectional view of a cell potential measuring device that includes the cell electrophysiological sensor in accordance with this exemplary embodiment.

This exemplary embodiment mainly differs from the first exemplary embodiment in that pillars 6 are formed in the vicinity of through-hole 1, i.e. in positions nearer to through-hole 1 than inner wall surface 3a of frame 3 and on diaphragm 2.

In this exemplary embodiment, pillars 6 are formed in the vicinity of through-hole 1. This structure can suppress the occurrence of bubbles in the vicinity of through-hole 1 and efficiently remove the bubbles when a liquid in the form of droplets is injected into cavity 4, using a dispenser, for example.

That is, in this exemplary embodiment, when a droplet made of extracellular fluid 10 makes contact with end surface 5 of frame 3, the tip of the droplet first makes contact with the tips of pillars 6 by means of the surface tension of extracellular fluid 10 forming the droplet. Then, while wetting outer wall surfaces 6a of pillars 6, the liquid made of extracellular fluid 10 is guided to through-hole 1 inside of cavity 4. At this time, bubbles escape between outer wall surfaces 6a of pillars 6 and inner wall surface 3a of frame 3 in the upward direction, and the liquid made of extracellular fluid 10 can be accurately charged in the vicinity of through-hole 1 together with cells 9 without bubbles occurring therein. Further, while moving along wall surfaces 6a of pillars 6, the remaining bubbles can be removed easily.

Preferably, each pillar 6 has a sectional shape equal to or larger than 100 μm in order to ensure a sufficient mechanical strength. When the spacing between adjacent pillars 6 is set to 50 to 100 μm, the liquid can permeate efficiently by means of the surface tension, and the advantage of pillars 6 can be maximized. That is, when the spacing between pillars 6 is smaller than 50 μm, the liquid is difficult to flow between pillars 6, and bubbles easily remain between pillars 6 and frame 3. When the spacing between pillars 6 is larger than 100 μm, the efficiency of the liquid permeation is reduced.

When pillars 6 are formed in positions farther from inner wall surface 3a of frame 3 and nearer to through-hole 1, the liquid can permeate toward through-hole 1 more efficiently. However, in order to secure a space for placing cells in the vicinity of through-hole 1, it is preferable to set the spacing between through-hole 1 and each pillar 6 to 50 μm or larger.

Further, when a plurality of pillars 6 are formed in a radial configuration so as to encircle through-hole 1, the above action can be enhanced. That is, this structure can ensure the contact of a part of the droplet with a part of any one of pillars 6 even when the tip of the dispenser, such as a micropipette, tilts or the droplet has a less symmetrical shape. Thus this structure allows the liquid, such as extracellular fluid 10, to be charged while reliably suppressing the occurrence of bubbles from wall surfaces 6a of pillars 6 formed in the vicinity of through-hole 1 and considerably reducing remaining bubbles.

Further, imparting hydrophilic properties to the surfaces of pillars 6 can enhance wettability to the liquid. Thereby, the occurrence of bubbles can be suppressed and the bubbles can be removed more effectively.

In order to impart hydrophilic properties to the surfaces of wall surfaces 6a of pillars 6, it is preferable to remove carbon molecules adhering to the surfaces so that the surfaces are made clean. Immediately thereafter, the frame is stored in a container filled with pure water. Thereby, the cleanliness of the surfaces can be maintained. Preferably, the hydrophilic properties at this time are such that the contact angle is equal to or smaller than 10°. This contact angle can be measured by applying a droplet of de-ionized pure water to the surface of an object to be measured.

Further, as another means for enhancing hydrophilic properties, coating the surfaces of wall surfaces 6a of pillars 6 with an insulating film of silicon dioxide, for example, is effective.

More preferably, all the surfaces to be in contact with the liquid, e.g. the inner wall surface of cavity 4, inner wall surfaces of diaphragm 2 and through-hole 1, are made hydrophilic. This structure can enhance wettability to the liquid for various measurements, thereby suppressing the occurrence of bubbles.

Further, when a plurality of pillars 6 are formed at spacings having different dimensions, pillars 6 facilitating the escape of bubbles and pillars 6 facilitating permeation of the liquid can be mixed.

In this manner, in the cell electrophysiological sensor of this exemplary embodiment, pillars 6 formed in the vicinity of through-hole 1 allow the gas, such as air, present inside of cavity 4 to be easily released to the outside from pillars 6. At the same time, while wetting wall surfaces 6a of pillars 6, the liquid can be promptly charged into cavity 4 without bubbles in the vicinity of through-hole 1. As a result, this structure allows the cell electrophysiological sensor to have improved measuring reliability. Further, imparting hydrophilic properties to wall surfaces 6a enhances the wettability of pillars 6 to the liquid, and the liquid can be charged into cavity 4 promptly. At the same time, the liquid can be charged without bubbles in the vicinity of through-hole 1.

As shown in FIG. 9, the cell potential measuring device including the cell electrophysiological sensor of this exemplary embodiment allows the liquid to be charged without remaining bubbles, gives a chemical stimulation, for example, to a specimen cell, and measures current or voltage between the reference electrode and the measuring electrode.

In this manner, the method for measuring electrophysiological activity occurring during the activity of cells 9 is the same as that described in the first exemplary embodiment, and thus the description thereof is omitted.

As described above, in this exemplary embodiment, first, a droplet containing cells 9 and dispensed from a dispenser makes contact with a part of the nearest pillar 6. Then, the droplet moves on wall surface 6a forming this pillar 6 by means of the surface tension of the liquid. Thereafter, in the vicinity of frame 3, the liquid can be guided to the center of diaphragm 2 where through-hole 1 is present. Thus the occurrence of bubbles can be suppressed and remaining bubbles can be removed efficiently.

### Fifth Exemplary Embodiment

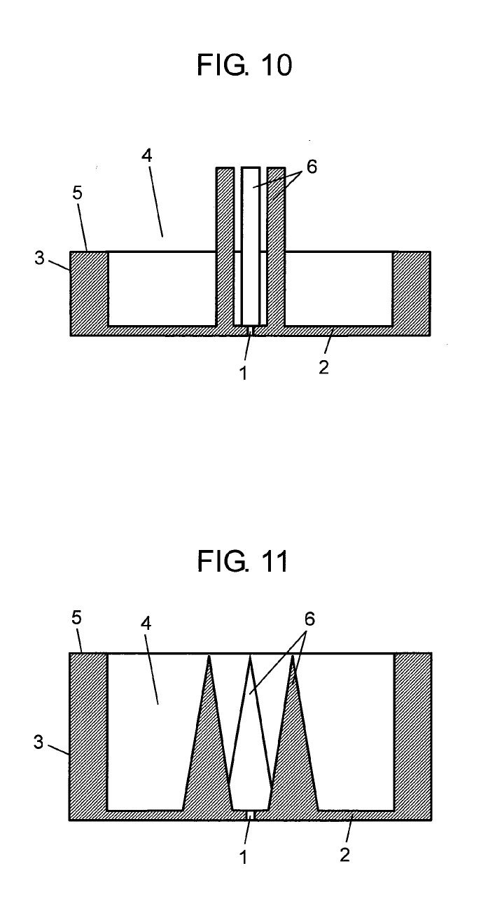

A description is provided for a cell electrophysiological sensor in accordance with the fifth exemplary embodiment of the present invention with reference to FIG. 10. FIG. 10 is a sectional view of the cell electrophysiological sensor in accordance with the fifth exemplary embodiment of the present invention.

The structure of the cell electrophysiological sensor of this exemplary embodiment largely differs from that of the cell electrophysiological sensor of the fourth exemplary embodiment of FIG. 7 in that the height of pillar 6 is greater than the height of end surface 5 of frame 3.

This structure can ensure the contact of the tip of a droplet with the tips of pillars 6 and thus reliably suppress remaining bubbles even when a device of which tip has a large aperture, such as a micropipette, is used.

In this exemplary embodiment, pillars 6 and diaphragm 2 are unitarily formed, and thus pillars 6 having a greater height in this manner have a high mechanical strength.

### Sixth Exemplary Embodiment

A description is provided for a cell electrophysiological sensor in accordance with the sixth exemplary embodiment of the present invention with reference to FIG. 11. FIG. 11 is a sectional view of the cell electrophysiological sensor in accordance with the sixth exemplary embodiment of the present invention.

The structure of the cell electrophysiological sensor of this exemplary embodiment largely differs from that of the cell electrophysiological sensor of the fourth exemplary embodiment of FIG. 7 in that each pillar 6 has a conical shape and its tip has an acicular shape formed with an acute angle.

In this exemplary embodiment, no bubbles remain at the tip of each pillar 6. Even when pillars 6 are designed to have a small spacing therebetween, a space where the liquid diffuses can be provided on the tip side of each pillar 6. This is considered to be for the following reason: when the tip of pillar 6 exerts a large stress on a part of a droplet, it is difficult for the droplet to have surface tension, which increases the affinity of the droplet with outer wall surface 6a of pillar 6.

That is, in this exemplary embodiment, the surface tension of the droplet is distributed, and thus the liquid can be easily guided between pillars 6. Therefore, remaining of bubbles in the vicinity of through-hole 1 can be suppressed.

### Seventh Exemplary Embodiment

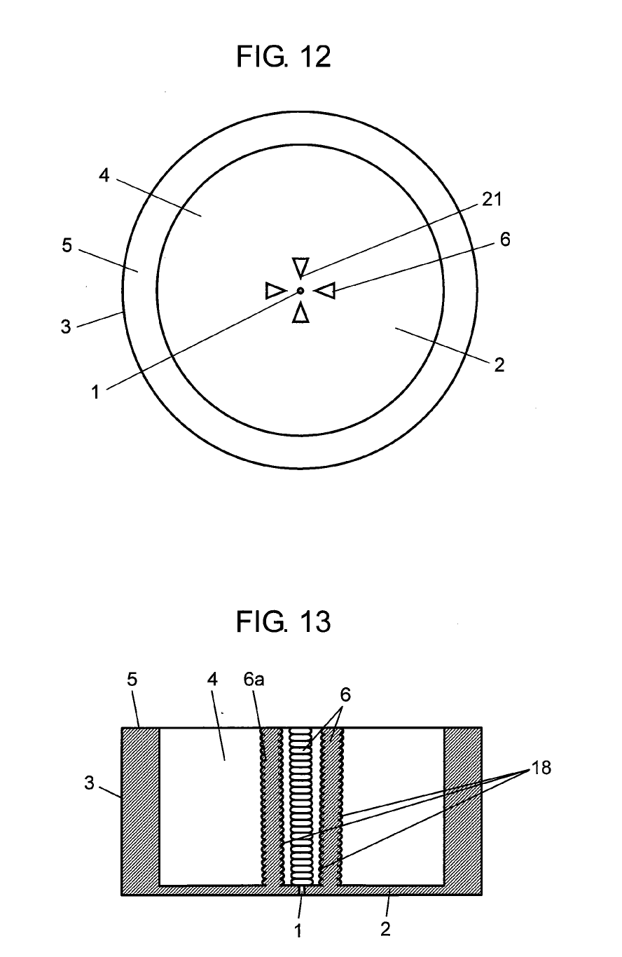

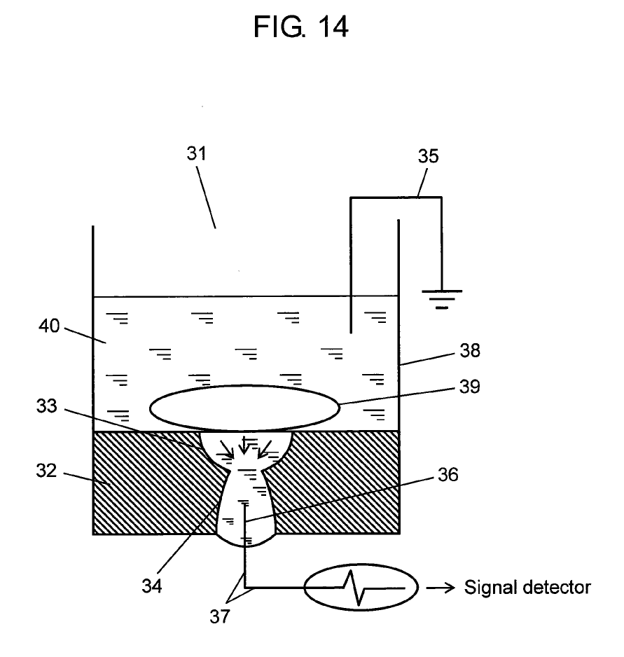

A description is provided for a cell electrophysiological sensor in accordance with the seventh exemplary embodiment of the present invention with reference to FIG. 12.

FIG. 12 is a top view of the cell electrophysiological sensor in accordance with the seventh exemplary embodiment of the present invention. The structure of the cell electrophysiological sensor of this exemplary embodiment largely differs from that of the cell electrophysiological sensor of the fourth exemplary embodiment of FIG. 7 in that pillars 6 each having a polygonal sectional shape are formed so that ridge lines 21 thereof face through-hole 1.

This structure can enhance the wettability to the liquid flowing from well layer 15 or baffle plate 14 into cavity 4 and makes the escape of bubbles from the inner circumferential parts of pillars 6 more effective.

That is, the remaining bubbles can be reduced and the liquid can be made to flow more smoothly.

Further, this structure allows cells 9 to fall to the vicinity of through-hole 1 efficiently. Thus, even in a small number of cells 9, specimen cell 9a can be fixed onto through-hole 1 promptly.

### Eighth Exemplary Embodiment

A description is provided for a cell electrophysiological sensor in accordance with the eighth exemplary embodiment of the present invention with reference to FIG. 13. FIG. 13 is a sectional view of the cell electrophysiological sensor in accordance with the eighth exemplary embodiment of the present invention.

This exemplary embodiment mainly differs from the fourth exemplary embodiment of FIG. 7 in the following point: as shown in FIG. 13, a plurality of annular projections and depressions 18 are formed on outer wall surface (surface) 6a of each pillar along its side surface. These projections and depressions 18 can be formed by dry etching.

In this manner, projections and depressions 18 substantially perpendicular to the long axis direction of pillar 6 are formed on outer wall surface 6a of the pillar. With this structure, a droplet can be spread to the entire outer circumference of outer wall surface 6a of the pillar along these projections and depressions 18.

Though not shown in FIG. 13, similar to the third exemplary embodiment, grooves in the long axis direction may be formed on outer wall surface 6a of pillar 6. When the grooves are formed, an injected droplet can be guided to the vicinity of through-hole 1 along the grooves, and the occurrence of bubbles can be suppressed. This structure allows a cell electrophysiological sensor to have high measuring reliability.

When the grooves are formed, projections and depressions 18 intersecting with these grooves have arc shapes that lack parts of annular shapes. With this structure, while spreading to the outer circumference of pillars 6 along projections and depressions 18, the liquid can be efficiently guided to the side of diaphragm 2 along the intersecting grooves.

In this exemplary embodiment, through-hole 1, diaphragm 2, frame 3, pillars 6, and projections and depressions 18 on outer wall surfaces 6a of these pillars 6 can be formed in one process with high productivity.

In each of the above first through eighth exemplary embodiments, a cell electrophysiological sensor is described as an example of a biosensor. The present invention can be applied to various biosensors for measuring other specimens, such as a DNA, RNA, protein, amino acid, lipid membrane, carbohydrate, ion, antigen, or the like. In these cases, the specimen holder is a receptor, e.g. a DNA probe, an electrode, an antigen, or the like.

### INDUSTRIAL APPLICABILITY

The biosensor of the present invention is capable of suppressing the occurrence of bubbles in the vicinity of a through-hole and easily removing the remaining bubbles. Thus the measuring reliability of the cell electrophysiological sensor can be improved. For this reason, the present invention is highly useful as a biosensor in a medical field, for example, where high-accuracy measurement is required.

1. A biosensor comprising:
a diaphragm having a specimen holder;
a frame supporting the diaphragm and having a cavity; and
a pillar formed on a side of an inner wall surface of the frame and on a surface of the diaphragm.

2. The biosensor of claim 1, wherein the pillar is formed in contact with the inner wall surface of the frame so that an outer wall surface of the pillar forms an acute angle with the inner wall surface of the frame.

3. The biosensor of claim 2, wherein a sectional shape of the pillar is a polygonal shape and a ridge line of the polygonal shape is in contact with the inner wall surface of the frame.

4. The biosensor of claim 1, wherein the pillar is formed nearer to the specimen holder than an inner wall of the frame.

5. The biosensor of claim 4, wherein the pillar is one of a plurality of pillars formed so as to encircle the specimen holder.

6. The biosensor of claim 4, wherein a sectional shape of the pillar is a polygonal shape and a ridge line of the polygonal shape faces the specimen holder.

7. The biosensor of claim 4, wherein the pillar is one of a plurality of pillars and a spacing between the adjacent pillars ranges 50 to 100 μm inclusive.

8. The biosensor of claim 4, wherein a height of the pillar is greater than a height of the frame.

9. The biosensor of claim 1, wherein an outer wall surface of the pillar is hydrophilic.

10. The biosensor of claim 1, wherein the pillar is unitarily formed with an inner wall of the frame.

11. The biosensor of claim 1, wherein a plurality of projections and depressions are formed on a surface of the pillar in a direction intersecting with a long axis direction of the pillar.

12. The biosensor of claim 1, wherein a plurality of grooves are formed on a surface of the pillar in a long axis direction of the pillar.

13. The biosensor of claim 1, wherein a height of the pillar is equal to a height of the frame.

14. The biosensor of claim 1, wherein a sectional shape of the pillar is a polygonal shape.

15. The biosensor of claim 1, wherein the pillar and the diaphragm are unitarily formed.
